(19) United States
(12) Patent Application Publication
(10) Pub. No.: US 2023/0368911 A1
(43) Pub. Date: Nov. 16, 2023

(54) Contactless Patient Monitoring System

(71) Applicant: FGC Holdings Limited, North York (CA)

(72) Inventors: David Ponevac, Miami Beach, FL (US); Alaa Alkadi, Jacksonville, FL (US); Peter Friedmann, Toronto (CA)

(73) Assignee: FGC Holdings Limited, North York (CA)

(21) Appl. No.: 18/305,814

(22) Filed: Apr. 24, 2023

Related U.S. Application Data
(60) Provisional application No. 63/333,662, filed on Apr. 22, 2022.

(51) Int. Cl. G16H 40/67; G16H 40/20; G16H 15/00; A61B 5/00

(52) U.S. Cl. CPC G16H 40/67; G16H 40/20; G16H 15/00; A61B 5/6889; A61B 5/7282; A61B 5/7435; A61B 5/746

(57) ABSTRACT

A contactless patient monitoring system is provided and includes a central processing unit, a sensor array, and a web-based central interface. The sensor array is positioned on a ceiling of a room and establishes a link with the central processing unit. The sensor array includes a plurality of sensors, which when triggered, sends data to the central processing unit for analyzing the data. The central processing unit issues an actionable event depending on which sensor of the plurality of sensors in triggered. The web-based central interface receives the actionable event and displays a report on a user dashboard.

### CROSS-REFERENCE TO RELATED APPLICATIONS

The application claims priority to U.S. Application No. 63/333,662, filed on Apr. 22, 2022.

### FIELD OF THE INVENTION

The invention relates to a patient monitoring system and, more particularly, to a fully integrated contactless patient monitoring system.

### BACKGROUND

Over the years, the monitoring technology has evolved and developed with aiding and assisting caregivers in protecting and serving a patient while for example, in an assisted living, hospital, or during home care. However, these systems have had their individual drawbacks and a system that could provide increased aid in situational awareness for a caregiver, as well as improved responsiveness and coverage of monitoring would be beneficial. A system that provides these benefits would give a caregiver the tools he or she needs to increase their efficiency and overall care of the patient. Further, the system would provide the patient and the patient's family better peace of mind knowing that if there is an emergency or a patient needs something, the caregiver will be alerted right away.

Therefore, there is currently a need for a system to provide better support for a patient and provide more assistance to a caregiver.

### SUMMARY

A contactless patient monitoring system is provided and includes a central processing unit, a sensor array, and a web-based central interface. The sensor array is positioned on a ceiling of a room and establishes a link with the central processing unit.

The sensor array includes a plurality of sensors, which when triggered, sends data to the central processing unit for analyzing the data. The central processing unit issues an actionable event depending on which sensor of the plurality of sensors in triggered. The web-based central interface receives the actionable event and displays a report on a user dashboard.

### BRIEF DESCRIPTION OF DRAWINGS

The invention will now be described by way of example with reference to the accompanying Figures of which:

FIG. 1 is a schematic diagram of a contactless patient monitoring system according to the invention;

FIG. 2 is a diagram view of the dashboard screen of the contactless patient monitoring system according to the invention;

FIG. 3 is a diagram view of the alarm manager screen of the contactless patient monitoring system according to the invention;

FIG. 4 is a diagram view of the music library screen for the contactless patient monitoring system according to the invention;

FIG. 5 is a perspective view of one possible embodiment of the communication(s) gateway of the contactless patient monitoring system according to the invention;

FIG. 6 is a top, front perspective view of the bottom cover of the sensor array of FIG. 5;

FIG. 7 is a top, front perspective view of the lower level of the sensor array of FIG. 6;

FIG. 8 is a top, front perspective view of the upper level of the sensor array of FIG. 7; and

FIG. 9 is a top, front perspective view of the top cover of the sensor array of FIG. 8.

### DETAILED DESCRIPTION OF THE EMBODIMENT(S)

In an embodiment, the teachings herein describe a system that allows for the creation of a contactless patient monitoring system 1.

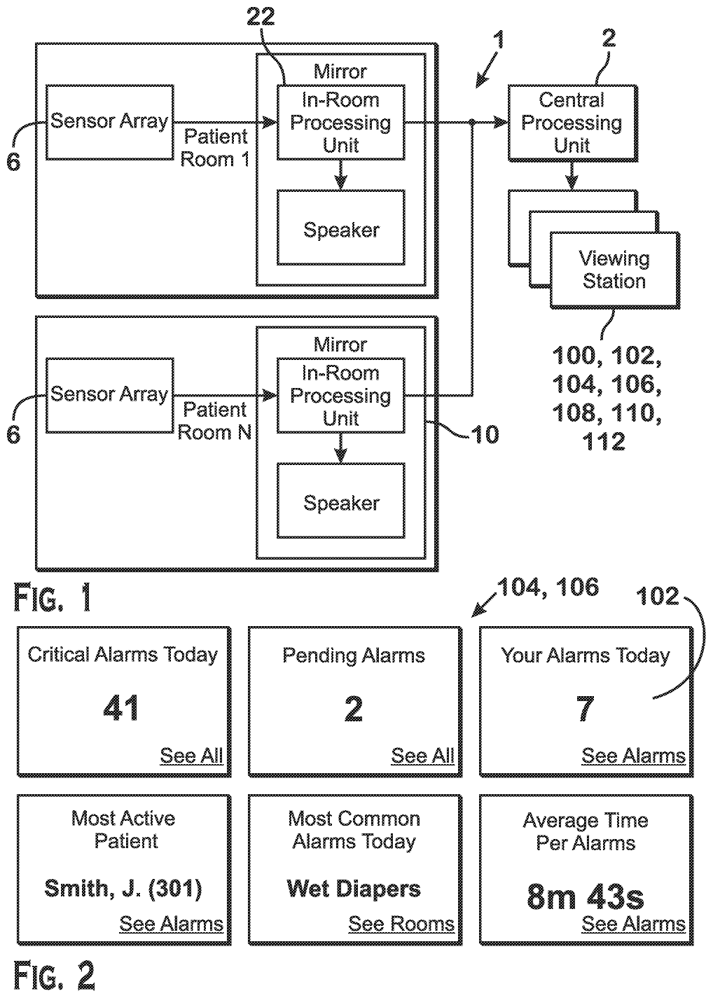

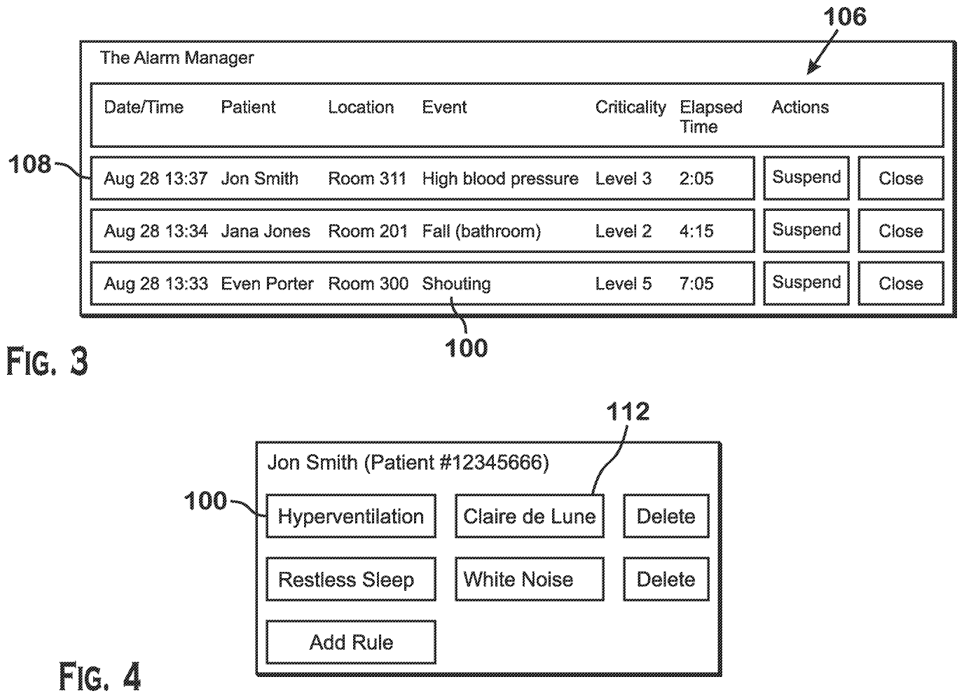

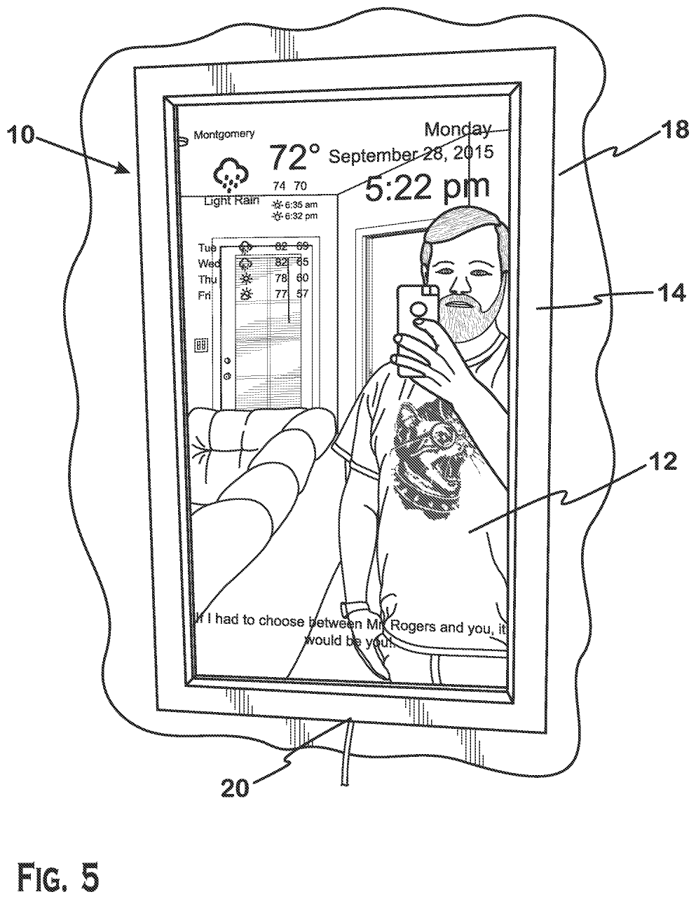

In the exemplary embodiment, the contactless patient monitoring system 1 is shown in FIGS. 1-9. In the exemplary embodiment, the contactless patient monitoring system 1 generally includes a central processing unit 2, a communication(s) gateway 4 and a sensor array 6.

In the exemplary embodiment, the central processing unit 2 generally includes the following major components: a plurality of service nodes. The service node is a standard service node. One of ordinary skill in the art would understand the inner workings of the service node and applicant's design is not the exclusive embodiment.

The service node of the central processing unit 2 is able to both communicate with other service nodes or function in isolation with only the central processing unit 2.

In the exemplary embodiment, the central processing unit 2 further operates on an operating system, for example, Linux, Windows or other OSes. However, one of ordinary skill in the art would understand the applicant's design is not the exclusive embodiment.

In the exemplary embodiment, the central processing unit 2 further includes a plurality of graphics processing unit cards which are utilized by the central processing unit 2 to analyze the incoming data and generate detections from a stream. The graphics processing unit cards are standard graphic processing unit cards. One of ordinary skill in the art would understand the inner workings of the graphic processing unit card and understand the applicant's design is not the exclusive embodiment.

In one embodiment, the communication(s) gateway 4 provides for a human interaction element between a patient and a caregiver. One of ordinary skill in the art would understand the applicant's design is not the exclusive embodiment. The communication(s) gateway 4 has the ability to form a network link between a sensor array 6 and the central processing unit 2. One of ordinary skill in the art would understand the applicant's design is not the exclusive embodiment.

In one embodiment, an example of the communication(s) gateway 4 is a smart mirror 10 as shown in FIG. 5. One of ordinary skill in the art would understand the applicant's design is not the exclusive embodiment. In one exemplary embodiment, the smart mirror 10 generally includes a mirror lens 12, a frame housing 14, a plurality of through-holes 20 and an in-room processing unit 22.

As illustrated, the mirror lens 12 is a standard mirror lens with a semi-transparent surface. One of ordinary skill in the art would understand the applicant's design of the mirror lens 12 is not the exclusive embodiment.

In the exemplary embodiment, the frame housing 14 generally includes a mirror edging 16, and an elongated rear cover 18. In the exemplary embodiment, the mirror edging 16 is a standard mirror edging. One of ordinary skill in the art would understand the applicant's design of the mirror edging is not the exclusive embodiment. In the exemplary embodiment, the elongated rear cover 18 is a rectangular shell-like member. One of ordinary skill in the art would understand the applicant's design is not the exclusive embodiment.

In the exemplary embodiment, the in-room processing unit 22 is a standard processing unit. One of ordinary skill in the art would understand the inner workings of the in-room processing unit and the applicant's design is not the exclusive embodiment.

The smart mirror 10 is optional. The communication(s) gateway 4 can be utilized in different design formats as a communication platform. One skilled in the art would understand the applicant's design is not the exclusive embodiment.

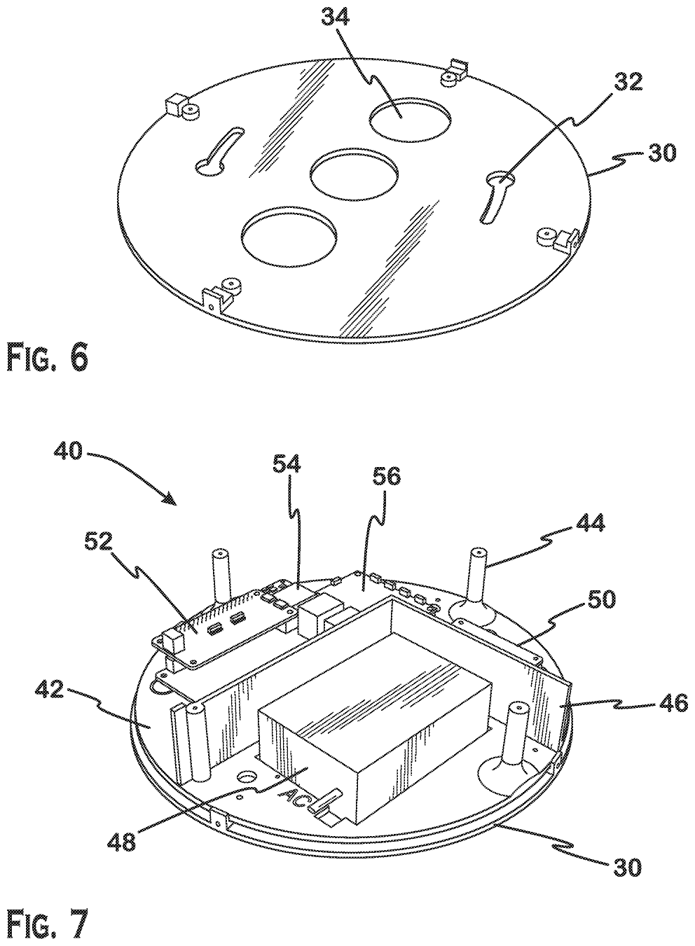

In the exemplary embodiment, the sensor array 6 generally includes a bottom cover 30, a lower level 40, an upper level 60, and a top cover 80 as shown in FIGS. 6-9.

In the exemplary embodiment, the bottom cover 30 generally includes a plurality of ceiling mount holes 32 as shown in FIG. 6. Each ceiling mount hole 32 is an elongated cylindrical member and bulbous at one end. One of ordinary skill in the art would understand the applicant's design is not the exclusive embodiment.

In the exemplary embodiment, the bottom cover 30 further includes a plurality of wiring harness spaces 34. The wiring harness spaces 34 are circular and located in the central region of the bottom cover 30. One of ordinary skill in the art would understand the applicant's design is not the exclusive embodiment.

In the exemplary embodiment, the lower level 40 generally includes a mount 42 as shown in FIG. 7. In the exemplary embodiment, the mount 42 is a standard mount for securing electronic components. One of ordinary skill in the art would understand the applicant's design is not the exclusive embodiment.

As illustrated, the mount 42 further includes a plurality of connectors 44. The plurality of connectors 44 are protruding cylindrical members located around the perimeter of the mount 42. One of ordinary skill in the art would understand the applicant's design is not the exclusive embodiment.

As illustrated, the mount 42 further includes a divider 46. The divider 46 is a plate like member forming an angle and extending to the perimeter of the mount 42.

As shown, the lower level 40 further includes a power supply unit 48. The power supply unit 48 is a standard power supply. One of ordinary skill in the art would understand the inner workings of a power supply unit 48.

As illustrated, the lower level 40 further includes a power delivery circuit board 50. The power delivery circuit board 50 is a standard power delivery circuit board. One of ordinary skill in the art would understand the inner workings of a power delivery circuit board 50.

As illustrated, the lower level 40 further includes a network enabled microprocessor unit 52. The microprocessor unit 52 is a standard microprocessor unit. One of ordinary skill in the art would understand the inner workings of the microprocessor unit 52.

As shown, the lower level 40 further includes a sound amplifier circuit 54. The sound amplifier circuit 54 is a standard sound amplifier circuit.

In the exemplary embodiment, the lower level 40 further includes a sensor distribution board 56. The sensor distribution board 56 is a standard distribution board. One of ordinary skill in the art would understand the inner workings of the sensor distribution board and the applicant's design is not the exclusive embodiment.

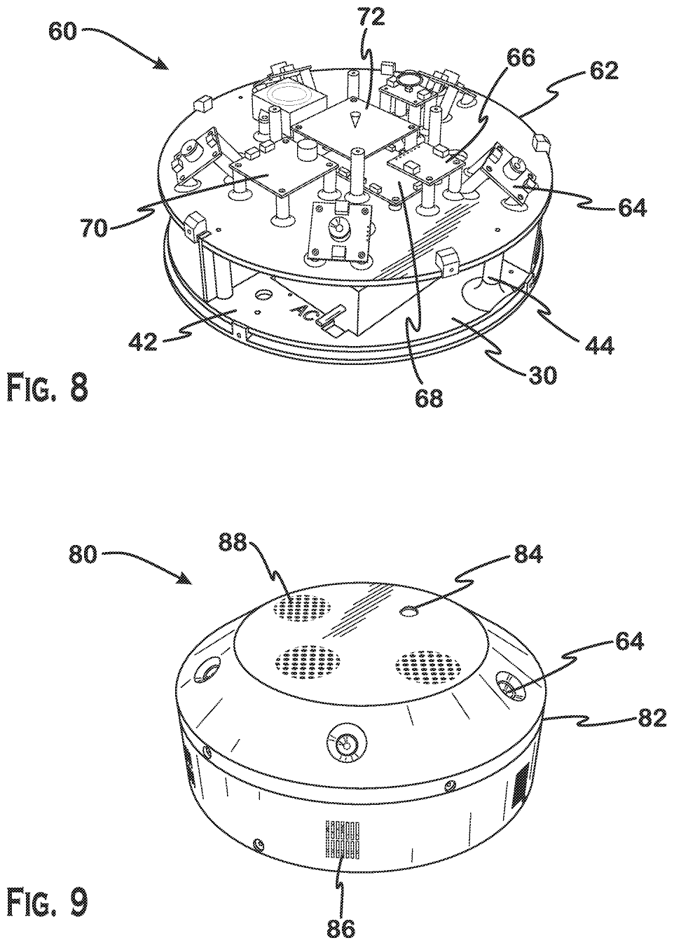

In the exemplary embodiment, the upper level 60 generally includes a mount 62 as shown in FIG. 8.

The mount 62 is a circular member for securing electronic components. One of ordinary skill in the art would understand the applicant's design is not the exclusive embodiment.

As shown, the upper level 60 further includes a plurality of thermal measurement units 64. The thermal measurement unit 64 is a standard thermal measurement unit.

As illustrated, the upper level 60 further includes an environment multi-sensor 66. The environment multi-sensor 66 is a standard environment multi sensor. One of ordinary skill in the art would understand the inner workings of an environment multi-sensor 66 and the applicant's design is not the exclusive embodiment.

As illustrated, the upper level 60 further includes a sensor distribution circuit 68. The sensor distribution circuit 68 is a standard distribution circuit.

As shown, the upper level 60 further includes a sound level measurement device 70. The sound level measurement device 70 is a standard sound level measurement device

As illustrated, the upper level 60 further includes a radio frequency device 72. The radio frequency device 72 is a standard radio frequency device.

In the exemplary embodiment, the top cover 80 generally includes an outer layer 82 as shown in FIG. 9.

As shown, the outer layer 82 is a shell-like member. One of ordinary skill in the art would understand the applicant's design is not the exclusive embodiment.

As illustrated, the outer layer 82 further includes a light sensor lens 84 positioned at a top of the outer layer 82. The light sensor lens 84 is a standard light sensor lens.

In the exemplary embodiment, the outer layer 82 further includes a plurality of cooling vents 86. The cooling vent 86 is circular shaped with a cross thread design. One of ordinary skill in the art would understand the applicant's design is not the exclusive embodiment.

As shown, the outer layer 82 further includes a plurality of sound and sensor vents 88. The sound and sensor vent 88 is rectangular shaped with a vertical thread design. One of ordinary skill in the art would understand the applicant's design is not the exclusive embodiment.

In the exemplary embodiment, the contactless patient monitoring system 1 generally includes the following major components: a central processing unit 2, a communication(s) gateway 4 and a sensor array 6.

The central processing unit 2 is either cloud-based or hosted on a facility network within range of the plurality of in-room processing units 22 and the sensor arrays 6. One of ordinary skill in the art would understand the applicant's design is not the exclusive embodiment.

In one embodiment, when the central processing unit is cloud-based, the central processing unit 2 sends data to a cloud which is segmented into different facility stations in order to not mix customer data across different facilities.

In another embodiment, when the central processing unit is hosted on a facility network the central processing unit 2 become the receiver and the data from either the in-room processing unit 22 and the sensor array 6 is transfer to the central processing unit 2.

In one embodiment of the communication(s) gateway 4 and the smart mirror 10 is utilized.

As shown, the smart mirror's 10 mirror lens 12 is inserted into the mirror edging 16. The in-room processing unit 22 is inserted inside the elongated rear cover 18 of the smart mirror 10. The mirror edging 16 of the smart mirror 10 is fastened to the elongated rear cover 18. The elongated rear cover 18 is further mounted to a wall or propped on a stand.

The sensor array 6 is positioned on the ceiling of a room and establishes a link with the in-room processing unit. In this embodiment, the sensor array 6 sends all collected data to the in-room processing unit 22 which sends the data to the central processing unit 2 for analyzing.

In the exemplary embodiment, the communication gate way 4 is not utilized. The sensor array 6 is positioned on the ceiling of a room and establishes a link directly with the central processing unit which sends data to the central processing unit 2 for analyzing.

In either embodiment, the sensor array 6 is associated with a specific room so the central processing unit 2 communicates and is able to determine which room the detection was made.

In the exemplary embodiment, the power supply unit 48, the power delivery circuit board 50, the microprocessor unit 52, the sounds amplifier circuit 54 and the sensor distribution board 56 are fastened to the mount 42 of the lower level 40 of the sensor array 6.

The mount 62 of upper level 60 of the sensor array 6 is fastened to the plurality of connectors 44 of the lower level 40 of the sensor array 6.

The plurality of thermal measurement units 64, the environment multi-sensor 66, the sensor distribution circuit 68, the sound level measurement device 70 and the radio frequency device 72 are fastened to the mount 62 of the upper level 60 of the sensor array 6.

The top cover 80 of the sensor array 6 encloses the upper level 60 and the lower level 40 of the sensor array 6 and is fastened to the bottom cover 30 of the sensor array 6.

In one embodiment, a network connection is made between the central processing unit 2 and the in-room processing unit 22 (which is housed inside the communication(s) gateway 4.

In another embodiment, a network connection is made between the central processing unit 2 and the sensor array 6. The sensor array may directly link with the central processing unit 2 through conventional networking such as Wifi or an ethernet cord. Whether the communication(s) gateway 4 would be utilized in establishing a connection between the central processing unit 2 and the sensor array 6 would depend on the user's installation configuration.

In either embodiment, the network link is established between the sensor array 6/in-room processing unit 22 and the central processing unit 2 by a transmission control protocol/internet protocol (TCP/IP). Each sensor array 6/in-room processing unit 22 is configured with an address of the central processing unit 2 in order to form the connection. During start up, the sensor array 6/in-room processing unit 22 will reach out to a preconfigured central processing unit 2 internet protocol (IP) address. In case the central processing unit 2 is down or not reachable, the sensor array 6/in-room processing unit 22 will periodically retry to establish the connection.

In the exemplary embodiment, the sensor array 6 is positioned within a patient's room. The sensor array 6 includes a plurality of sensors which are active and can be triggered depending on which situation arises. The thermal measurement unit 64 of the sensor array 6 tracks the presence of at least one person and can measure a person's body temperature. The environmental multi sensor 66 of the sensor array 6 takes measurements such as air pressure, humidity, temperature, volatile organic compound (VOC) level, Carbon Dioxide (CO2) level, dew point, etc. The light sensor lens 84 of the sensor array 6 measures the brightness of a room in lux. The sound level measurement device 70 of the sensor array 6 measures audio frequencies and analyzes the frequencies for known audio identifiers such as screams, gunshots, glass shattering, falls, etc. The radio frequency device 72 of the sensor array 6 measures and quantifies the spatial data in the RF spectrum by tracking reflected energy of an object or living being. The radio frequency device 72 further includes the ability to monitor the respirations of a patient during sleep if there are no other outside movements taking place.

When a sensor is triggered, the sensor array 6 relays the signal to either to the in-room processing unit 22, which the in-room processing unit 22 then again relays the signal to the central processing unit 2 or sensor array will send the signal directly to the central processing unit 2. In either scenario, the sensor array 6 or the in-room processing unit utilize a representational state transfer (REST)-based application programming interface (API) to communicate with the central processing unit 2 in a predefined intervals.

The communication(s) gateway 4 is an optional feature which can further allow an immediate interaction between the caregiver and patient. The communication(s) gateway 4 is not needed as stated and the sensor array 6 is able to directly send an alert to the central processing unit 2 and bypass the communication(s) gateway 4.

The central processing unit 2 receives the signal from the sensor array 6 or the in-room processing unit 22 and triggers an actionable event 100 depending on what is triggered in the sensor array 6.

The central processing unit 2 analyzes the incoming data and triggers the proper detection response. Upon receipt of the raw and preprocessed sensor data from sensor arrays 6, the central processing unit 2 first stores the sensor data in a database. Then a series of background processes take the unprocessed data one by one and put it through a series of algorithms. The analysis of the central processing unit 2 provides a series of steps through which the data flows and during each step a series of questions is asked by the central processing unit 2 which determines a confidence score. Essentially, data is compared, analyzed, matched, and filtered into different forms until a conclusion is reached.

For each of the algorithms utilized, the central processing unit 2 generates a probability score of how likely it is that a certain alarm condition has been met. A load balancing scheme is employed to efficiently distribute the data across multiple graphic processing unit cards in order to speed up the detection process. An output of these algorithms leads to alarms.

For example, a stream of CO readings flow from the sensor array 6 to the central processing unit 2. There the data is cleaned to eliminate spurious readings and if a safe threshold of parts per million of CO gas is exceeded, an alarm 102 is sent. The central processing unit 2 examines sensory data and generate a response based on for example: the patient, the caregiver, the patient's residence or the facility. The data is accessible using a web-based central interface 104. When an alarm is sent, it is directed to a web-based central interface 104 and displayed on a dashboard 106 which includes a plurality of user-configurable layouts. Moreover, the data can be accessible through mobile application as well as other embedded platforms depending on the user's preference.

The dashboard 106 offers the user the ability to take a quick glimpse of what is happening within their facility or in a particular patient's room, in real time. Further, the user can access general trends depending on the user's preferred time frame. Moreover, the dashboard 106 can show general trends such as any pending alarms, displaying relevant statistics, etc.

The dashboard 106 further includes an alarm manager 108 which is a central message que of all events that need caregiver attention. Determining which events require human attention would be based on the caregiver or the company.

Once an alarm 102 is triggered, a indicator is displayed on a queue-like user interface on the alarm manager 108 where it must be processed by a person and acknowledged.

The life span of the alarm 102 goes through several states—from new, to active, to overdue, to closed, and to archived. The alarm 102 is considered new when the sensor array 6 triggers the signal. The alarm 102 is considered active when the alarm 102 has been acknowledged. The alarm 102 will stay active until the situation is handled and then the indicator is cleared off the screen. The alarm 102 is considered overdue when the alarm 102 has not been acknowledged. The alarm 102 is considered closed when the alarm 102 has been acknowledged and resolved which then moves the alarm 102 to a storable table prior to being archived. The alarm 102 is considered archived when the alarm 102 is moved into archive for audit trail purposes. Additionally, the central processing unit 2 learns from the different alarm 102 conditions by an algorithm which reduces the false positive rate and increases overall performance.

The alarm manager 108 provides both historical archive of all processed events as well as an audit trail of which events occurred, how fast the alert was handled and who handled the alert. The alarm manager 108 displays at least the following information: the date and time of the event, the name of the patient, the location of the patient, the description of the event, the criticality of the event, the elapsed time of the event (from the moment the alarm was activated to when the alarm was terminated), etc. Further, the caregiver can interact with the alarm manager 108 such as suspend the alarm, close the alarm, or add additional information or notes.

Once the data is processed, the alarm manager 108 data is sent to a reporting manager 110. The reporting manager 110 permits the caregiver or company to create a variety of reports such as the alarm type, the frequency of the alarm, the frequency of the event, etc. Further, the report can be created in the time frame needed by the user. Reports are generated from the data available in the alarm queue paired together with all the other relevant metadata tied to that alarm (raw data, graphs, images, staff notes, etc.). When the report or series of reports is requested, the report managers pool all the data together from several different tables and generates one cohesive report where all of the data is viewable in an easy to understand format. Back-end of the reporting manager is done by structured query language (SQL) at the database level and then the reports manager formats it into a PDF file.

The central processing unit 2 further includes a large library of music 112 which can be associated with each individual patient or triggered by a specific actionable event set by the caregiver. This could be used as a calming method for the patient to reduce stress levels from a high stress situation and/or to let the patient know the caregiver is on the way. When the caregiver determines the proper protocol for handling a certain situation, the central processing unit 2 relays the signal back to either the in-room processing 22 or directly to the sensor array 6 which interprets the signal.

The foregoing illustrates some of the possibilities for practicing the invention. Many other embodiments are possible within the scope and spirit of the invention. It is, therefore intended that the foregoing description be regarded as illustrative rather than limiting, and that the scope of the invention is given by the appended claims together with their full range of equivalents.

What is claimed is:

1. A contactless patient monitoring system comprising:
a central processing unit;
a sensor array is positioned on a ceiling of a room and establishes a link with the central processing unit, the sensor array includes a plurality of sensors which when triggered sends data to the central processing unit for analyzing the data, the central processing unit issues an actionable event depending on which sensor of the plurality of sensors in triggered; and
a web-based central interface receives the actionable event and displays a report on a user dashboard.

2. The contactless patient monitoring system of claim 1, wherein the user dashboard includes an alarm manager.

3. The contactless patient monitoring system of claim 2, wherein once an alarm is triggered, a indicator is displayed on a queue-like user interface on the alarm manager.

4. The contactless patient monitoring system of claim 3, wherein the plurality of sensors includes a thermal measurement unit.

5. The contactless patient monitoring system of claim 4, wherein the plurality of sensors further includes an environment multi-sensor.

6. The contactless patient monitoring system of claim 5, wherein the plurality of sensors further includes a radio frequency device.

7. The contactless patient monitoring system of claim 6, wherein the plurality of sensors further includes a light sensor lens.

8. The contactless patient monitoring system of claim 7, wherein the plurality of sensors further includes a sound level measurement device.

9. The contactless patient monitoring system of claim 8, wherein the alarm includes a plurality of states.

10. The contactless patient monitoring system of claim 9, wherein the alarm includes a new state when a sensor of the plurality of sensors is triggered.

11. The contactless patient monitoring system of claim 10, wherein the alarm includes an active state where the alarm is acknowledged a user.

12. The contactless patient monitoring system of claim 11, wherein the alarm includes an overdue state.

13. The contactless patient monitoring system of claim 12, wherein the alarm includes an archived state when the alarm is moved into an archive of the alarm manager.

14. The contactless patient monitoring system of claim 13, wherein the central processing unit further comprises a reporting manager.

15. The contactless patient monitoring system of claim 14, wherein the central processing unit further comprises a large library of music associated with an individual patient or triggered by a specific actional event.

16. The contactless patient monitoring system of claim 15, wherein the central processing unit relays a signal to the sensor array to transmit a proper song or noise from the large library of music.

17. A contactless patient monitoring system comprising:
a central processing unit;
a communication(s) gateway having an in-room processing unit; and
a sensor array connected to the central processing unit by the communications gateway, the sensor array includes a plurality of sensors which when triggered sends data to the in-room processing unit prior to sending the data to the central processing unit for analyzing the data by an algorithm which provides a plurality of steps, each step of the plurality of steps includes a series of questions that determines a confidence score of whether a certain alarm condition is satisfied.

18. The contactless patient monitoring system of claim 17, wherein the contactless patient monitoring system further comprises a web-based central interface.

19. The contactless patient monitoring system of claim 18, wherein the central processing unit sends the web-based central interface an actionable event.

20. The contactless patient monitoring system of claim 19, wherein the actionable event is displayed on a dashboard of the web-based central interface.

21. The contactless patient monitoring system of claim 20, wherein the dashboard includes an alarm manager.

22. The contactless patient monitoring system of claim 21, wherein the communication(s) gateway is a smart mirror.

23. The contactless patient monitoring system of claim 22, wherein the central processing unit further comprises a reporting manager.

24. The contactless patient monitoring system of claim 23, wherein the central processing unit further comprises a large library of music associated with an individual patient or triggered by a specific actional event.

25. The contactless patient monitoring system of claim 24, wherein the central processing unit relays a signal to the in-room processing unit transmit a proper song or noise from the large library of music.
